(19) United States
(12) Patent Application Publication
(10) Pub. No.: US 2003/0171706 A1
(43) Pub. Date: Sep. 11, 2003

(54) Adjustable size ankle brace

(76) Inventor: Ronald E. Nelson, Cambridge, MN (US)

Correspondence Address: FOLEY & LARDNER, 150 EAST GILMAN STREET, P.O. BOX 1497, MADISON, WI, 53701-1497, US

(21) Appl. No.: 10/095,366

(22) Filed: Mar. 11, 2002

(51) Int. Cl. A61F 13/00; A61F 13/06

(52) U.S. Cl. 602/65

(57) ABSTRACT

An ankle brace that can be configured to fit feet and ankles of differing sizes. The brace includes a base that wraps around the sides and rear of the foot and ankle, with forward edges that come toward another but do not meet. Two movable flaps are releasably fixed to the base, one on each side of the base, using hook and loop type fastening material. Straps and loops are mounted in alternating fashion on the forward edges of the two flaps, with one end of each strap remaining free. The free end of each strap can be trained through a loop mounted on the opposite movable flap and then looped back for attachment to the mounted end of the strap using hook and loop type fastening material, to fasten the brace securely to the foot and ankle. By repositioning the movable straps the effective size of the brace is varied.

### FIELD OF THE INVENTION

This invention relates generally to the field of articles worn by persons to reduce the likelihood, severity, or exacerbation of injury to the body, and more specifically to the field of braces worn on the ankle.

### BACKGROUND OF THE INVENTION

The ankle joint connects the lower leg and the foot of a person, providing a pivot point that allows the foot to rotate relative to the lower leg. Together the lower leg, ankle joint, and foot make up a complex system that must be sturdy yet flexible in order to bear a person's weight while providing freedom of movement.

The ankle joint is one of the most frequently used joints in the body, as it is required for any activity that involves walking or running. Further, it receives relatively high levels of stress as it bears dynamic loads that are multiples of the entire weight of the body. During ambulation, and especially during strenuous sports such as football, basketball, tennis, or soccer, quick changes in direction or uneven playing surfaces can cause the ankle to move beyond its normal range of motion, resulting in an injury commonly referred to as a sprained ankle. A sprained ankle is a source of some discomfort that can make sports less enjoyable or reduce athletic performance, and it can reduce mobility and productivity during normal day-to-day activities. For these reasons, there has long been motivation to find ways to support and protect the ankle while maintaining good freedom throughout the normal range of motion, to reduce injuries without diminishing mobility.

Ankle braces have been used for many years, in a variety of specific embodiments directed to particular applications. Although there is considerable variety in the construction of ankle braces today, one common feature is that eyelets and a shoelace are used to secure the ankle brace about the ankle joint and to adjust the degree of compression and tension provided by the brace. However, the eyelets and shoelace used for this purpose have several disadvantages.

First, shoelaces are known to break, and they may be most likely to do so in response to the kinds of stress encountered during vigorous sports. When a shoelace on an ankle brace breaks, the ankle joint may be subjected to unexpected stress, causing injury or exacerbating existing injury. For this reason, it would be useful to find a substitute for the shoelace and eyelets that would be less likely to undergo catastrophic failure.

Second, a shoelace threaded through eyelets and placed under tension by tying the shoelace will have essentially uniform tension throughout its length. If the eyelets are uniformly spaced, this means that the compression and tightness of the brace will be essentially uniform across all the pairs of eyelets. However, it may not be desirable to have uniform compression and tightness of the brace across all the pairs of eyelets. For example, it may be desirable to have greater compression above and below the ankle joint and lesser compression in the vicinity of the ankle joint in order to combine good support with adequate freedom of motion. It is known that by varying the spacing of the eyelets, the compression and tightness of the brace can be varied across the pairs of eyelets. However, once the brace is manufactured, the spacing of the eyelets is fixed, so that the compression and tightness of the brace cannot be adjusted to take into account the particular size and shape characteristics of the person wearing the brace. For this reason, it would be desirable to find a substitute for the shoelace and eyelets that would allow the compression and tightness of the brace to be flexibly adjusted across the length of the brace at the time the brace was fastened about the ankle joint, in order to take into account the particular size and shape characteristics of the person wearing the brace.

Third, a shoelace and eyelets require a fair degree of manual dexterity and the use of two hands when they are placed under tension and tied in order to fasten the brace about the ankle. Especially in contact sports where hands take physical punishment or where pads or protective equipment are worn, or in environments requiring gloves, manual dexterity may be limited so that the person wearing the brace may need the help of an additional person. Further, because of the high level of manual dexterity required, the process of fastening the brace may consume valuable time. For these reasons, it would be desirable to find a substitute for the shoelace and eyelets that would allow the brace to be fastened or adjusted with a lower level of manual dexterity.

### SUMMARY OF THE INVENTION

The present invention features an adjustable ankle brace in which fastening straps are used, rather than eyelets and a shoelace. The present invention features lower potential for catastrophic failure of the fastening about the ankle, and greater flexibility in adjustment of tension and tightness of fit about the ankle. The present invention also features easier fastening about the ankle compared to the prior art.

No matter how the strap is fastened, the tension applied by each strap is applied independently of the tension applied by other straps. Thus, if one strap breaks or becomes detached, the overall tension applied by the plurality of straps diminishes but is not lost completely, thereby avoiding the catastrophic failure associated with breakage of the shoelaces seen in the prior art. The tension applied by each strap can be adjusted independently of the tension applied by the other straps, thereby allowing the wearer to flexibly adjust the fit of the brace to accommodate a wide range of applications. Each strap can placed under tension using only one hand. This feature contrasts with the process of fastening the shoelaces used in the prior art, as tensioning and tying a shoelace requires the use of two hands and possibly even the assistance of another person. Further, the independence of the tension applied by each strap allows iterative adjustment of the fit, by starting out with low levels of tension and adjusting the tension of the straps to suit the wearer. The relatively large size of the straps used in this invention compared to the size of the shoelaces used in the prior art makes the straps relatively easy to grip, lowering the level of manual dexterity required to fasten or adjust the ankle brace.

The invention may utilize a base and a movable and detachable closure assembly. The closure assembly of the invention uses straps and loops to fasten the brace upon the foot and ankle. Alternatively, a different movable and detachable closure assembly may be used that has straps and fastening patches to fasten the brace upon the foot and ankle. In either case, the position of the closure assembly on the base can be changed by detaching and reattaching the closure assembly in a new position on the base to adjust the effective size of the brace to fit a wide range of sizes. If desired, closure assemblies may be utilized that are not movable and detachable.

Adjustable size ankle braces according to the invention may use hook and loop type fastening material as detachable attachment means. The straps, loops, and fastening patches may be placed along a curved line. Further, a resilient stay member may be mounted on each side of the base.

Further objects, features, and advantages of the invention will be apparent from the following detailed description when taken in conjunction with the accompanying drawings.

### BRIEF DESCRIPTION OF THE DRAWINGS

In the drawings:

FIG. 1 is a perspective view of an ankle brace according to the invention installed on a foot;

FIG. 2 is a perspective view of the base of the ankle brace;

FIG. 3 is a back view of the base of the ankle brace pressed flat;

FIG. 4 is a front view of a resilient stay member made of flattened springs.

FIG. 5 is a side view of a resilient stay member made of flattened springs.

FIG. 6 is a front view of the closure assembly of the ankle brace;

FIG. 7 is a back view of the closure assembly of the ankle brace;

FIG. 8 is a perspective view of the ankle brace with the detachable and movable side flaps of the closure assembly exploded from the base;

FIG. 9 is a perspective view of a modified embodiment of an ankle brace according to the invention installed on a foot;

FIG. 10 is a front view of the closure assembly of the ankle brace of FIG. 9;

FIG. 11 is a back view of the closure assembly of the ankle brace of FIG. 9;

FIG. 12 is a perspective view of the ankle brace of FIG. 9 with the detachable and movable side flaps of the closure assembly exploded from the base;

FIG. 13 is a perspective view of a further embodiment of an ankle brace according to the invention installed on a foot with the fastening straps fastened;

FIG. 14 is a perspective view of the ankle brace of FIG. 13 installed on a foot with the fastening straps unfastened;

FIG. 15 is a perspective view of a further modified ankle brace according to the invention installed on a foot with the fastening straps fastened; and

FIG. 16 is a perspective view of the ankle brace of FIG. 15 installed on a foot with the straps unfastened.

### DETAILED DESCRIPTION OF THE INVENTION

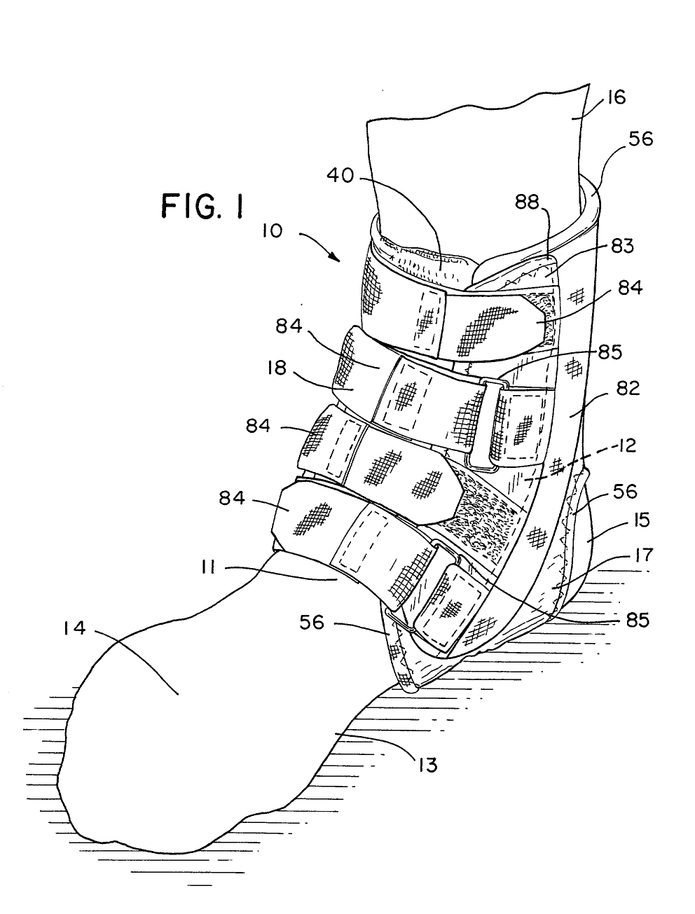

With reference to the drawings, FIG. 1 shows an ankle brace in accordance with the invention indicated generally at 10, as worn on the right foot 11 and ankle 12 of a person. Although the figures and this detailed description show the ankle brace worn on the right foot, the construction of the ankle brace is preferably essentially symmetric and the ankle brace can be worn on either the left or right foot. The foot has an instep region 13 and a toe region 14 that extend out from an opening in the front of the ankle brace. The foot also has a heel region 15 that extends out from a opening in the rear of the ankle brace. The lower leg 16 of the person extends out from an opening at the top of the ankle brace. The ankle brace of FIG. 1 is comprised of a base, indicated generally at 17, and a closure assembly, indicated generally at 18.

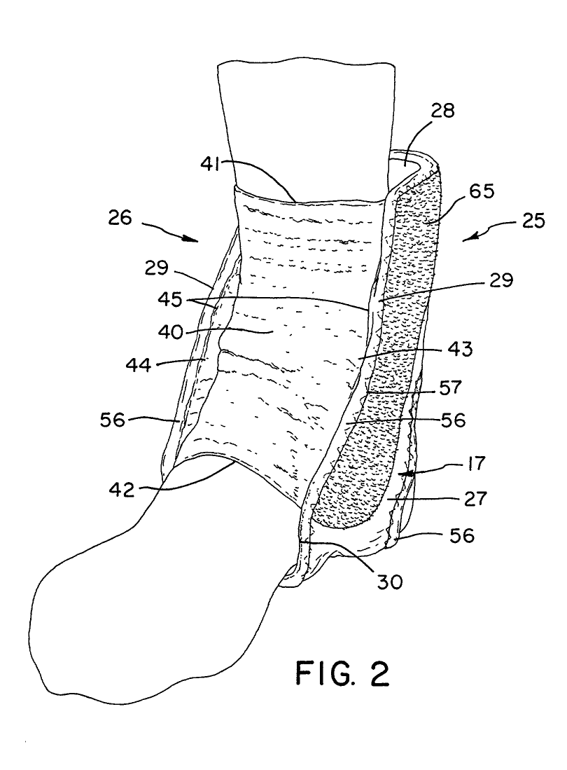

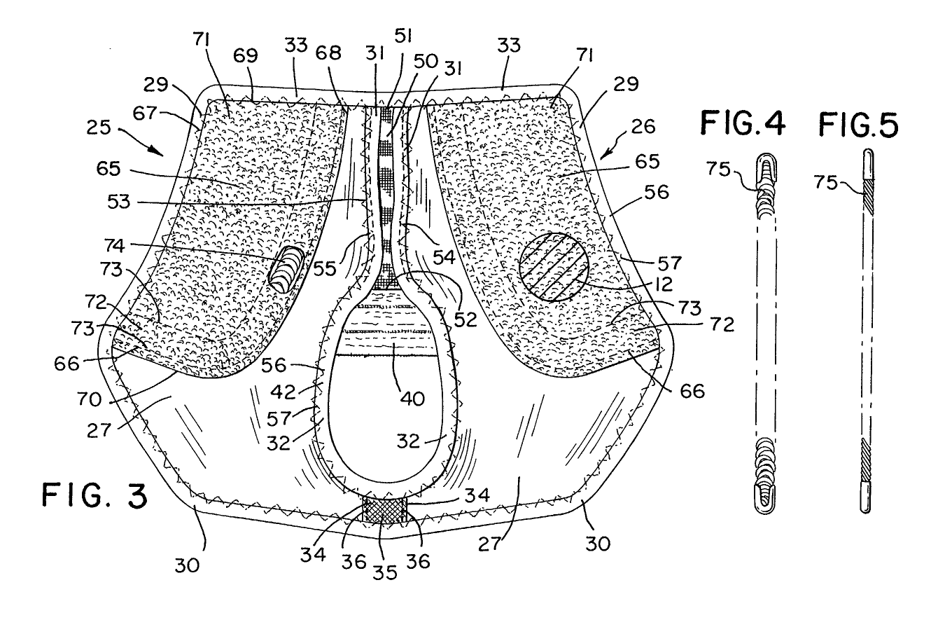

FIG. 2 shows the base 17 of the ankle brace 10 in perspective view. The base 17 has a first side 25 and a second side 26, each with an exterior surface 27 and a lining 28 that are formed of sheet materials. As shown in the back view of the base 17 in FIG. 3, the first side 25 and the second side 26 of the base 17 are preferably generally symmetrical. As best shown in FIG. 3, each side of the base has an upper forward edge 29, a lower forward edge 30, an upper rear edge 31, a lower rear edge 32, a top edge 33, and a bottom edge 34. Although the first side 25 and second side 26 of the base 17 may be made as a single piece, they are preferably made as two separate pieces, with a bottom edge attachment member 35 that is secured, preferably using stitching 36, to join the bottom edges 34 of the first and second sides of the base to each other so that the base wraps around the foot and ankle.

As best shown in FIG. 2, the ankle brace preferably has a front stretchable elastic member 40 that is approximately rectangular in shape with a top edge 41, a bottom edge 42 , a first side edge 43, and a second side edge 44. The first side edge 43 and the second side edge 44 of the front stretchable elastic member 40 are secured, preferably using stitching 45, to the upper forward edges 29 of the first side 25 and second side 26 of the base, so that the front stretchable elastic member 40 spans the gap between the upper forward edges 29 of the first and second sides of the base. This front stretchable elastic member 40 serves mainly to hold the base 17 on the ankle and foot during adjustment as the closure assembly 18 is the primary way that tension is applied to the brace to fasten it securely to the foot and ankle.

As best shown in FIG. 3, the ankle brace also preferably has a rear stretchable elastic member 50 that is approximately rectangular in shape with a top edge 51, a bottom edge 52, a first side edge 53, and a second side edge 54. The first side edge 53 and second side edge 54 of the rear stretchable elastic member 50 are secured, preferably using stitching 55, to the upper rear edges 31 of the first side 25 and second side 26 of the base, so that the rear stretchable elastic member 50 spans the gap between the upper rear edges 31 of the first and second sides of the base.

As best shown in FIG. 3, an edge binding 56 is secured, preferably using stitching 57, to cover the edges of the sides of the base (except for the bottom edges of the sides of the base 34 that are secured together with the bottom edge attachment member 35) and the top edge 51 of the rear stretchable elastic member 50. The lower forward edges 30 of the first side 25 and the second side 26 of the base and the bottom edge 42 of the front stretchable elastic member 40 form an opening for the toes. The lower rear edges 32 of the first side 25 and the second side 26 of the base and the bottom edge 52 of the rear stretchable elastic member 50 form an opening for the heel. The top edges 33 of the first side 25 and the second side 26 of the base, the top edge 41 (hidden in FIG. 3, shown in FIG. 2) of the front stretchable elastic member 40, and the top edge 51 of the rear stretchable elastic member 50 form an opening for the leg.

As best shown in FIG. 3, attachment means for detachable attachment of the closure assembly 18 to the base 17 is secured to the first side 25 and second side 26 of the base. The attachment means may be formed, for example, as mounting patches 65 that are secured to the first side 25 and second side 26 of the base, preferably using stitching 66. Each mounting patch 65 has a leading edge 67 near the upper forward edge 29 of the side of the base on which the patch is secured, a trailing edge 68 approximately parallel to and spaced substantially rearward of the leading edge 67, a top edge 69 near the top edge 33 of the side of the base on which the patch is secured, and a bottom edge 70 located near the bottom of the foot when the brace is worn. The mounting patches 65 are preferably of sufficient dimension to encompass the region on each side of the ankle 12. The exterior surface 71 of each mounting patch includes detachable attachment means, preferably comprised of material of the type that adheres when pressed against a complementary material, one example of which is the hook and loop type material sold under the trademark Velcro.

As best shown in FIG. 3, an elongate pocket 72 is preferably formed between each mounting patch 65 and the exterior surface 27 of the side of the base on which the mounting patch is secured, preferably by stitching 73. The stitching 73 may be, at least in part, the same stitching 66 used to secure the mounting patches 65 to the base, or it may be separate stitching. On each side, the elongate pocket 72 preferably runs approximately parallel to the trailing edge 68 and bottom edge 70 of the mounting patch 65, beginning at the upper trailing corner of the mounting patch where the top edge 69 and trailing edge 68 of the mounting patch meet, running parallel to the trailing edge 68 and the bottom edge 70 of the mounting patch, and ending at the lower leading corner of the mounting patch where the bottom edge 70 and leading edge 67 of the mounting patch meet. A resilient stay member 74 is preferably located in each elongate pocket 72. The resilient stay member may be formed of a pair of interleaved helical springs 75 made of stainless steel that have been flattened, as shown in FIGS. 4 and 5, or other flexible material of conventional construction commonly used in various types of braces. There may be more than one elongated side pocket 72 containing a resilient stay member 74 on each side, and the elongated side pocket 72 may be openable at one end to allow removal of the resilient stay member or replacement of the resilient stay member with a different resilient stay member having different resiliency to adjust the amount of support provided.

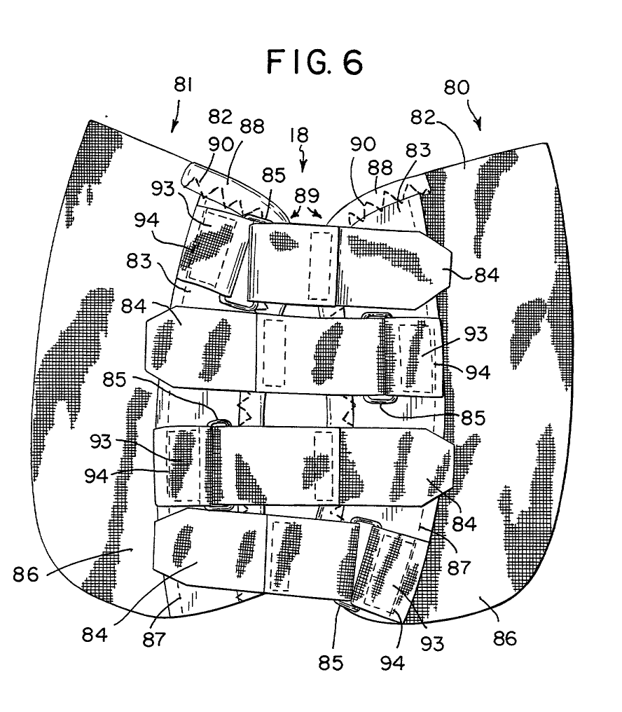

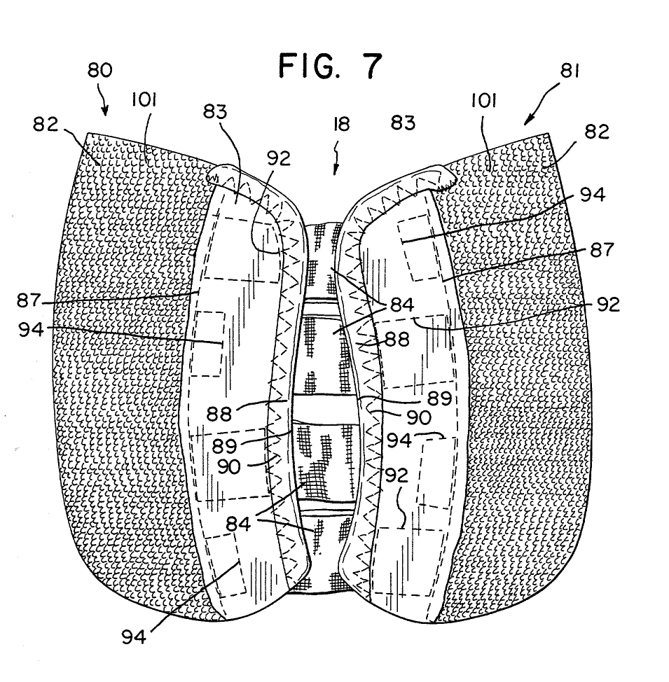

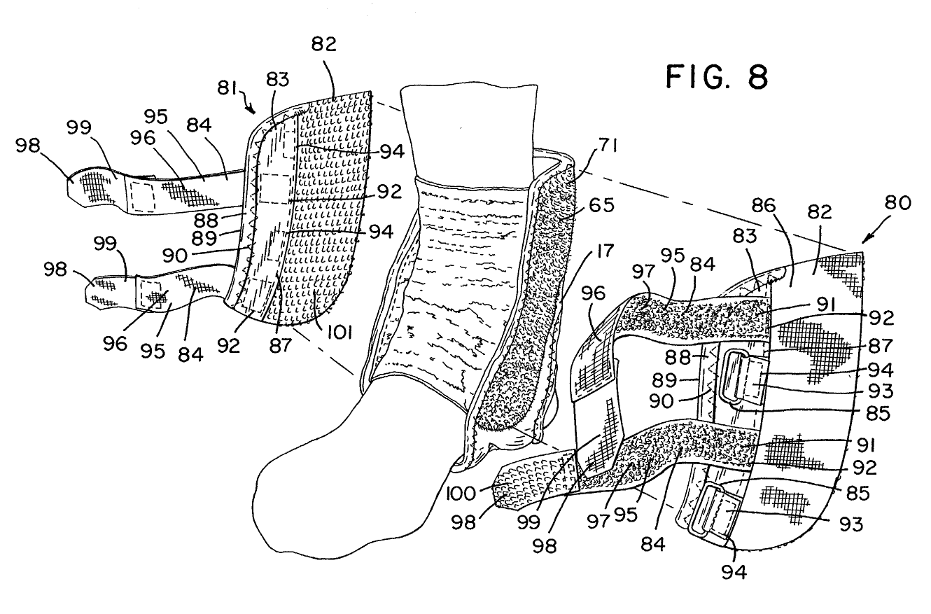

FIGS. 6 and 7 show front and back views of the closure assembly 18 in a closed position and without the base. FIG. 8 shows the closure assembly 18 in an open position exploded from the base. As best shown in FIG. 8, the closure assembly 18 has a first detachable movable side flap 80 and a second detachable movable side flap 81. Each detachable movable side flap is preferably comprised of an attachment tab 82, a carrier strip 83, fastening straps 84, and fastening loops 85. The carrier strip 83 is secured to the exterior surface 86 of the attachment tab, preferably using stitching 87. Edge binding 88 is preferably secured to the leading edge 89 of each detachable movable side flap, preferably using stitching 90.

As best shown in FIG. 8, each fastening strap 84 has a proximal end 91, that extends through and includes a middle section 95 of the strap, with an interior surface 96 and an exterior surface 97 and a distal end 98 with an interior surface 99 and an exterior surface 100. The proximal end 91 of each fastening strap 84 is secured to a detachable movable side flap at a location on a carrier strip 83, preferably using stitching 92. The exterior surface 97 of the proximal end 91 (including the middle section 95) and the exterior surface 100 of the distal end 98 include detachable attachment means, preferably comprised of materials of the type that adhere when pressed together, one example of which is the hook and loop type material sold under the trademark Velcro.

As best shown in FIG. 8, each fastening loop 85 is secured to a detachable movable side flap at a location on a carrier strip 83, preferably using a fabric loop 93 secured to the carrier strip 83 and attachment tab 82, preferably using stitching 94. Each fastening loop 85 is large enough that a fastening strap 84 may be passed through it. The fastening loops 85 are preferably made of metal, but they may be made of plastic, fabric, or other suitable material, or they may be implemented as slots in the detachable movable side flaps.

As best shown in FIG. 8, the fastening straps 84 and fastening loops 85 are arranged on the first detachable movable side flap 80 complementary to the arrangement on the second detachable movable side flap 81 so that a fastening strap 84 on one detachable movable side flap is opposite a fastening loop 85 on the other detachable movable side flap, preferably with an equal number of fastening straps 84 and fastening loops 85 on each detachable movable side flap arranged in alternating fashion along each carrier strip 83. Each fastening strap 84 may be trained through a fastening loop 85, and the distal end 98 of that fastening strap 84 detachably attached to at least a portion of its own proximal end 91 (which includes the middle section 95) by pressing the detachable attachment means on the exterior surface 100 of the distal end 98 against the detachable attachment means on the exterior surface 97 of the proximal end 91. In this way, tension may be placed on the fastening straps to fasten the ankle brace securely on the foot and ankle.

As best shown in FIG. 8, attachment means for detachable attachment of the closure assembly 18 to the base 17 is provided on the interior surface 101 of each attachment tab. The attachment means may be, for example, material of the type that adheres when pressed against a complementary material, one example of which is the hook and loop type material sold under the trademark Velcro. Each detachable movable side flap can be detachably attached to a side of the base 17 by pressing the attachment means found on the interior surface 101 of its attachment tab 82 against the attachment means found on the exterior surface 71 of a mounting patch 65. Once attached, the positions of the detachable movable side flaps relative to the base can be adjusted by pulling them away from the corresponding sides of the base, moving them relative to the base, and then re-attaching them. In this way, the size of the ankle brace can be adjusted to achieve a proper fit for a wide range of ankle and foot sizes.

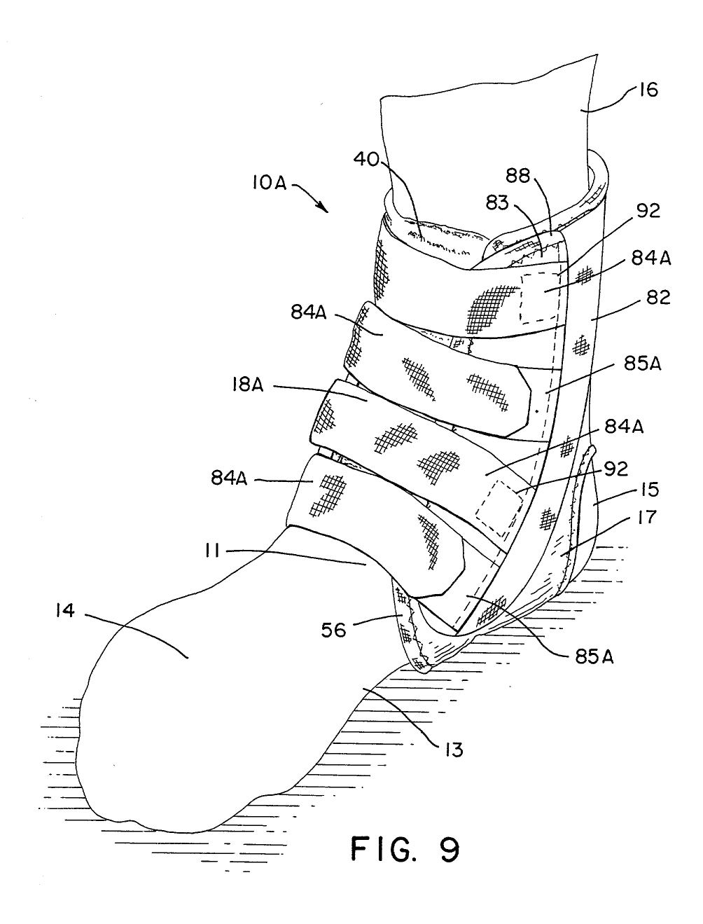

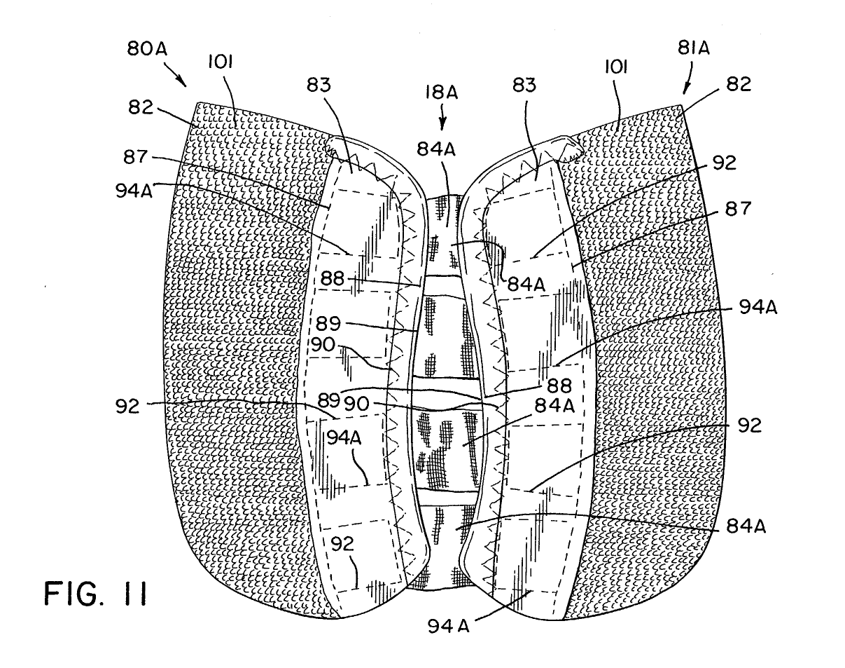

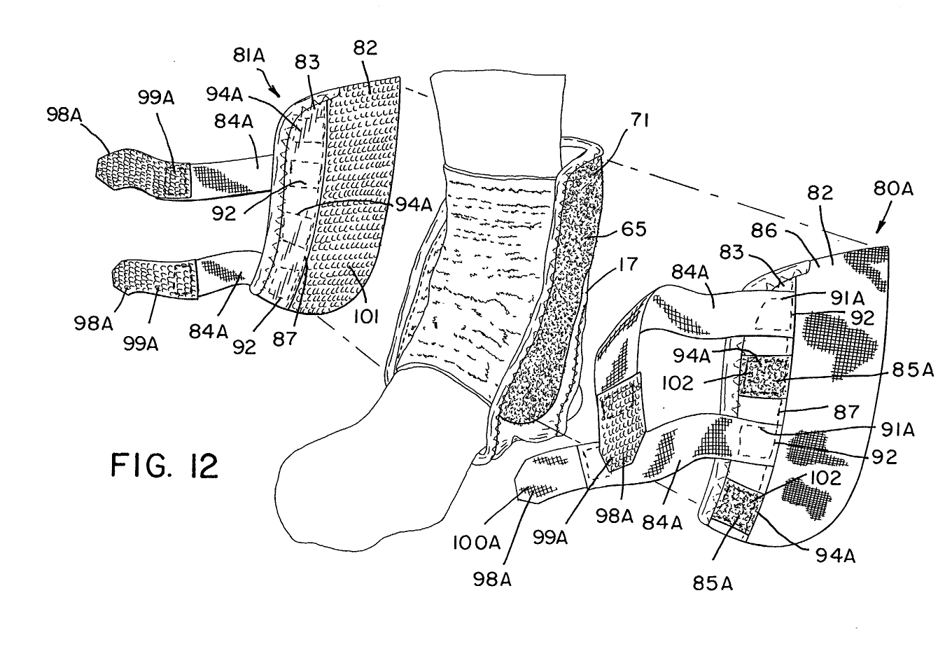

Although the ankle brace shown in FIGS. 1-8 uses a closure assembly with fastening straps and fastening loops that is detachably attached to the base, alternative embodiments of the invention may be used. Another embodiment of the ankle brace, shown in FIGS. 9-12, uses a different closure assembly 18A without fastening loops that is detachably attached to the same base 17 as is used in the ankle brace of FIGS. 1-8. Otherwise, the same parts perform the same functions as in the brace of FIGS. 1-8.

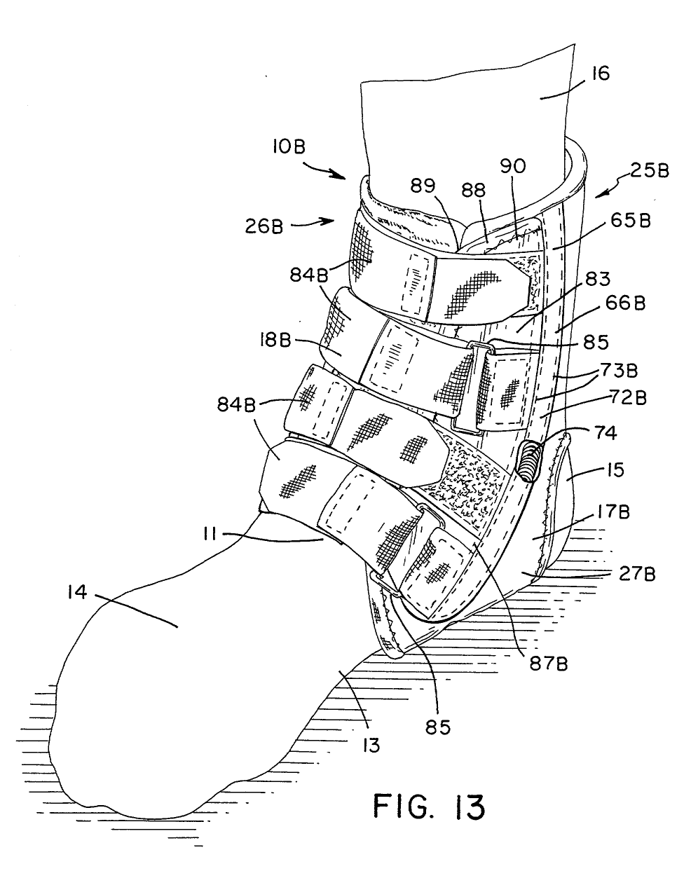

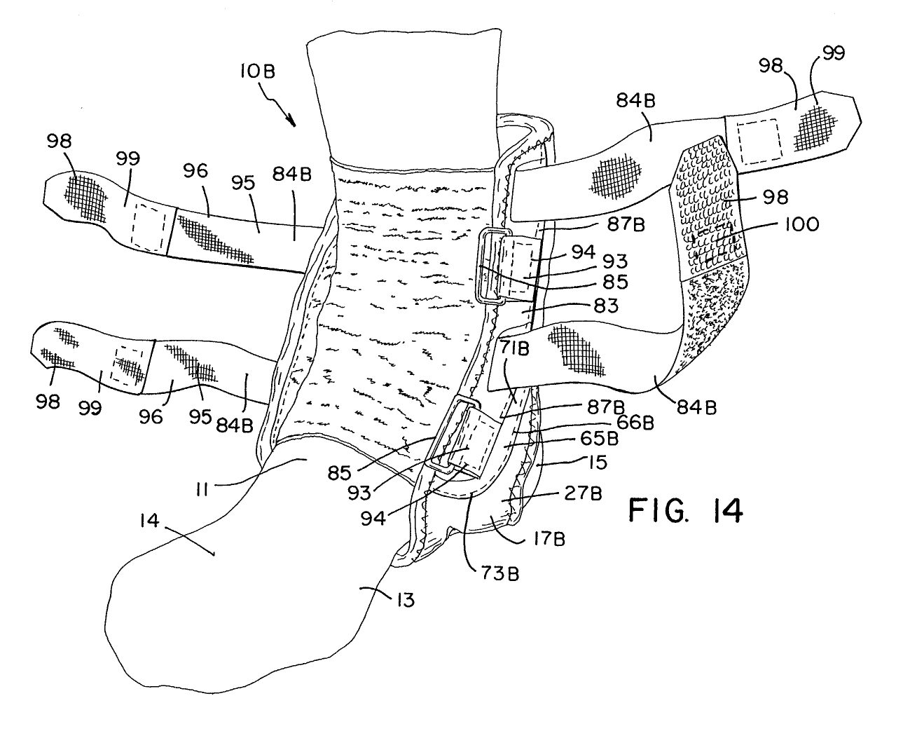

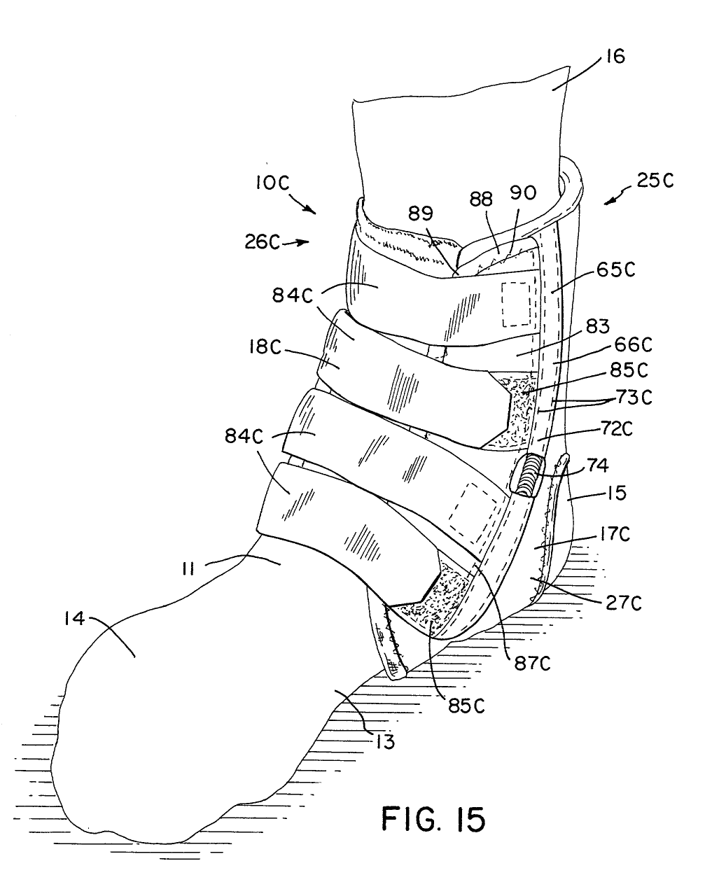

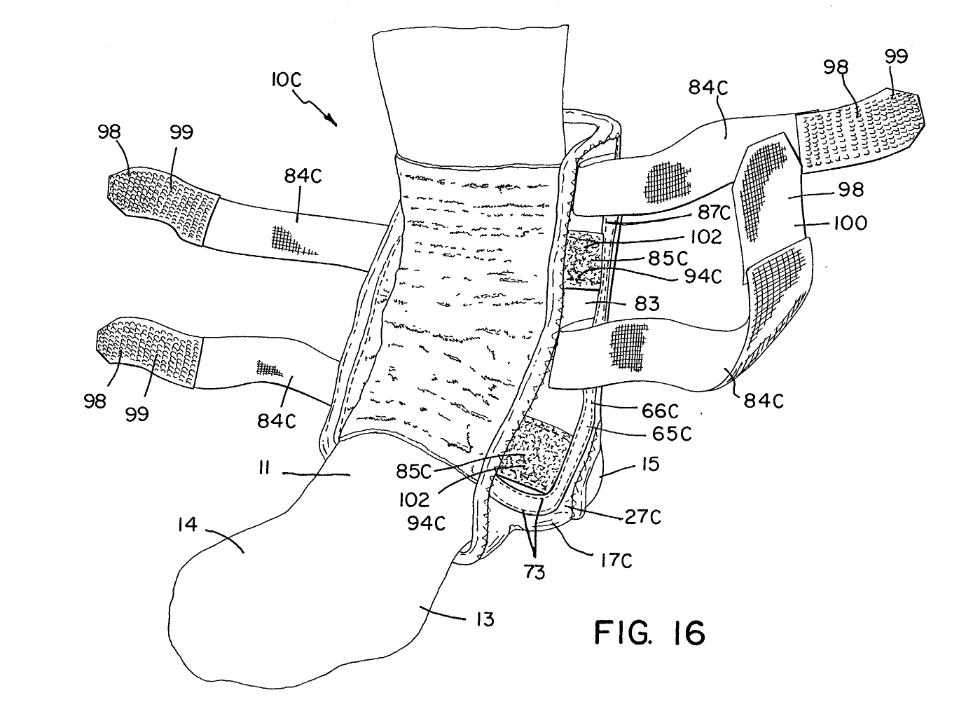

Further embodiments, shown in FIGS. 13-14 and in FIGS. 15-16, use closure assemblies 18B and 18C, respectively, similar to the ankle braces of FIGS. 1-8 and FIGS. 9-12, respectively, but the closure assemblies of the ankle braces of FIGS. 13-14 and FIGS. 15-16 are permanently secured to their bases using stitches instead of detachable attachment means, and are therefore not detachable. Instead of adjusting the size of the ankle brace by moving the closure assemblies, the fastening straps are adjusted to achieve a proper fit for a range of ankle and foot sizes.

As shown in FIGS. 9-12, the ankle brace 10A is similar to the ankle brace 10 of FIG. 1. In particular, the ankle brace 10A uses the same base 17 as the brace 10, but with a different closure assembly 18A. To avoid repetition of details which are the same for both embodiments, the following description of the ankle brace 10A will be concerned mainly with the details of the closure assembly 18A that differ from the details of the closure assembly 18 of the ankle brace 10.

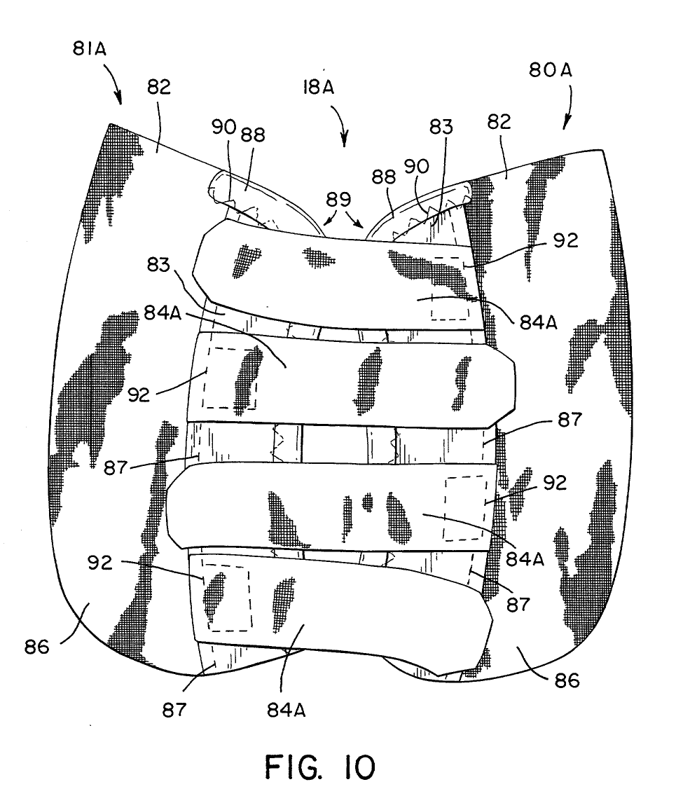

FIGS. 10, 11, and 12 show front, back, and exploded views of the closure assembly 18A of the ankle brace 10A. As best shown in FIG. 12, the closure assembly 18A has a first detachable movable side flap 80A and a second detachable movable side flap 81A. Each detachable movable side flap has an attachment tab 82 and a carrier strip 83 secured to the attachment tab, preferably using stitching 87. Fastening straps 84A and strap fastening patches 85A are secured in alternating fashion to each carrier strip 83, preferably using stitching 94A. Each strap fastening patch 85A is preferably approximately the same width as a fastening strap 84A and preferably approximately twice as long as it is wide. The fastening straps 84A and strap fastening patches 85A are arranged on the first detachable movable side flap 80A complementary to the arrangement on the second detachable movable side flap 81A so that a fastening strap 84A on one detachable movable side flap is opposite a strap fastening patch 85A on the other detachable movable side flap, preferably with an equal number of fastening straps 84A and strap fastening patches 85A on each detachable movable side flap.

As best shown in FIG. 12, each fastening strap 84A has a proximal end 91A and a distal end 98A with an interior surface 99A and an exterior surface 100A. The interior surface 99A of the distal end 98A and the exterior surface 102 of the strap fastening patch include detachable attachment means, that are preferably comprised of materials of the types that adhere when pressed together, one example of which is the hook and loop type material sold under the trademark Velcro. Tension may placed on each fastening strap 84A, and the detachable attachment means on the interior surface 99A of the distal end 98A of the fastening strap may be detachably attached to the detachable attachment means on a strap fastening patch 85A. In this way, the ankle brace according to the second preferred embodiment may be fastened securely on the foot and ankle.

As shown in FIG. 13, the ankle brace 10B is similar to the ankle brace 10 shown in FIG. 1. In particular, the ankle brace 10B uses a base 17B that is similar to the base 17, and a closure assembly 18B that is similar to the closure assembly 18. The main difference between the ankle brace 10B and the ankle brace 10 is that the closure assembly 18B is permanently secured to the base 17B, instead of being detachable. To avoid repetition of details which are the same for both embodiments, the following description of ankle brace 10B will be concerned mainly with those details that differ from those of the ankle brace 10.

FIG. 13 shows a perspective view of the ankle brace 10B as worn on the right foot and ankle of a person. The ankle brace 10B is comprised of a base, indicated generally at 17B, and a closure assembly, indicated generally at 18B, that is permanently secured to the base, preferably using stitches 66B. The base 17B has the same general construction as the base 17 of the first and second preferred embodiments, except that the mounting patches 65 found on the base 17 are replaced with reinforcing patches 65B, and the elongate pockets 72B are formed in a different way on base 17B of the third embodiment.

As best shown in FIG. 13, the ankel brace 10B has reinforcing patches 65B instead of the mounting patches 65 found on the ankle brace 10. The reinforcing patches 65B are approximately the same shape and size as the mounting patches 65, and the reinforcing patches 65B are secured to the first side 25B and second side 26B of the base 17B, preferably using stitching 66B. An elongate pocket 72B is preferably formed between each reinforcing patch 65B and the exterior surface 27B of the side of the base on which the reinforcing patch is secured, preferably by stitching 73B. The stitching 73B may be, at least in part, the same stitching 66B used to secure the reinforcing patches 65B to the base, or it may be separate stitching. A resilient stay member 74 (of the same type as the resilient stay member 74 on the base 17 of the first and second preferred embodiments) is preferably located in the elongate pocket 72B. The elongate pockets 72B are approximately the same size and shape as the elongate pockets 72 on the base 17 of ankle braces 10 and 10A, and are otherwise similar in that there may be more than one elongated side pocket 72B containing a resilient stay member 74 on each side, and the elongated side pocket 72B may be openable at one end to allow removal of the resilient stay member or replacement of the resilient stay member with a different resilient stay member having different resiliency to adjust the amount of support provided.

FIG. 13 shows the closure assembly 18B with the fastening straps fastened, and FIG. 14 shows the closure assembly with the fastening straps unfastened. The closure assembly 18B has the same general construction as the closure assembly 18, except that the closure assembly 18B does not have the attachment tab 82 (as the reinforcing patch 65B replaces both the attachment tab 82 on the closure assembly 18 and the mounting patch 65 found on the base 17) and the closure assembly 18B is not detachable.

As best shown in FIG. 14, the closure assembly 18B has a carrier strip 83, fastening straps 84B, and fastening loops 85. The carrier strip 83 is secured to the exterior surface 71B of the reinforcing patch 65B, preferably using stitching 87B. Closure assembly 18B is otherwise the same as the closure assembly 18.

As shown in FIG. 15, the ankle brace 10C is similar to the ankle brace 10A shown in FIG. 9. In particular, the ankle brace 10C uses a base 17C that is similar to the base 17, and a closure assembly 18C that is similar to the closure assembly 18A. The main difference between the ankle brace 10C and the ankle brace 10A is that the closure assembly 18C is permanently secured to the base 17C instead of being detachable. To avoid repetition of details which are the same for both embodiments, the following description of ankle brace 10C will be concerned mainly with those details that differ from those of ankle brace 10A.

FIG. 15 shows a perspective view of the ankle brace 10C, as worn on the right foot and ankle of a person. The ankle brace 10C is comprised of a base, indicated generally at 17C, and a closure assembly, indicated generally at 18C, that is permanently secured to the base, preferably using stitches 66C. The base 17C has the same general construction as the base 17, except that the mounting patches 65 found on the base 17 are replaced with reinforcing patches 65C, and the elongate pockets 72C are formed in a different way.

As best shown in FIG. 15 and similar to the ankle brace 10B, the ankle brace 10C has reinforcing patches 65C instead of the mounting patches 65. The reinforcing patches 65C are approximately the same shape and size as the mounting patches 65, and the reinforcing patches 65C are secured to the first side 25C and second side 26C of the base 17C, preferably using stitching 66C. An elongate pocket 72C is preferably formed between each reinforcing patch 65C and the exterior surface 27C of the side of the base on which the reinforcing patch is secured, preferably by stitching 73C. The stitching 73C may be, at least in part, the same stitching 66C used to secure the reinforcing patches 65C to the base, or it may be separate stitching. A resilient stay member 74 (of the same type as the resilient stay member 74 on the base 17 of ankle braces 10 and 10A) is preferably located in the elongate pocket 72C. The elongate pockets 72C are approximately the same size and shape as the elongate pockets 72 on ankle brace 10 and the elongate pockets 72B on ankle brace 10B, and are otherwise similar in that there may be more than one elongated side pocket 72C containing a resilient stay member 74 on each side, and the elongated side pocket 72C may be openable at one end to allow removal of the resilient stay member or replacement of the resilient stay member with a different resilient stay member having different resiliency to adjust the amount of support provided.

FIG. 15 shows the closure assembly 18C with the fastening straps fastened, and FIG. 16 shows the closure assembly with the fastening straps unfastened. The closure assembly 18C has the same general construction as the closure assembly 18A of the ankle brace 10B, except that the closure assembly 18C does not have the attachment tab 82 (as the reinforcing patch 65C replaces both the attachment tab 82 found on the closure assembly 18A and the mounting patch 65 found on the base 17), and the closure assembly 18C is not detachable.

As best shown in FIG. 16, the closure assembly 18C has a carrier strip 83, fastening straps 84C, and strap fastening patches 85C. The carrier strip 83 is secured to the exterior surface 71C of the reinforcing patch 65C, preferably using stitching 87C. Closure assembly 18C of ankle brace 10C is otherwise the same as the closure assembly 18A of the second embodiment.

It is understood that the invention is not confined to the embodiments set forth herein as illustrative, but embraces all such forms thereof as come within the scope of the following claims.

What is claimed is:

1. An adjustable size ankle brace, comprising:
(a) a base of flexible material shaped to wrap around the rear and sides of a foot and ankle, the base having a first side and a second side;
(b) a first detachable movable side flap for detachable attachment to the first side of the base, and a second detachable movable side flap for detachable attachment to the second side of the base;
(c) a first detachable attachment means for detachable attachment of the first detachable movable side flap to the first side of the base, and a second detachable attachment means for detachable attachment of the second removable flap to the second side of the base;
(d) a first plurality of straps and a second plurality of straps, each strap having a proximal strap end with an exterior surface, a third detachable attachment means on the exterior surface of the proximal strap end, a distal strap end with an exterior surface, and a fourth detachable attachment means on the exterior surface of the distal strap end;
(e) a first plurality of loops and a second plurality of loops;
(f) the proximal strap end of each strap in the first plurality of straps and each loop in the first plurality of loops mounted to the first detachable movable side flap, the proximal strap end of each strap in the second plurality of straps and each loop in the second plurality of loops mounted to the second detachable movable side flap the straps and loops mounted so that when the first detachable movable side flap is attached to the first side of the base and the second detachable movable side flap is attached to the second side of the base, each strap in said first plurality of straps is opposite, and trainable through, a loop in said second plurality of loops and each strap in said second plurality of straps is opposite, and trainable through, a loop in said first plurality of loops whereby the positioning of the detachable movable side flaps is adjustable by removing the detachable movable side flaps from the base and repositioning them on the base according to the size of the foot and ankle of the intended wearer of the brace, and the distal end of each strap may be trained through a loop and the exterior surface of the distal end of the strap may be detachably attached to the exterior surface of the proximal end of the strap to apply tension to the strap to secure the base about the foot and ankle.

2. The ankle brace of claim 1 wherein the first, second, third, and fourth detachable attachment means comprise hook and loop type synthetic fastening material.

3. The ankle brace of claim 1 wherein the loops and proximal strap ends are attached to the carrier strips along a line which is slightly curved in conformance with the curvature of the front superior surface of the foot and ankle.

4. The ankle brace of claim 1 further comprising elongated pockets on either side of the base, and a resilient stay member located in each elongate pocket.

5. An adjustable size ankle brace, comprising:
(a) a base of flexible material shaped to wrap around the rear and sides of a foot and ankle, the base having a first side and a second side;
(b) a first detachable movable side flap for detachable attachment to the first side of the base, and a second detachable movable side flap for detachable attachment to the second side of the base;
(c) a first detachable attachment means for detachable attachment of the first detachable movable side flap to the first side of the base, and a second detachable attachment means for detachable attachment of the second removable flap to the second side of the base;
(d) a first plurality of straps and a second plurality of straps, each strap having a proximal strap end, a distal strap end with an interior surface, and a third detachable attachment means on the interior surface of the distal strap end;
(e) a first plurality of strap fastening patches and a second plurality of strap fastening patches, each strap fastening patch having an exterior surface and a fourth detachable attachment means on the exterior surface of the strap fastening patch;
(f) the proximal strap end of each strap in the first plurality of straps and each strap fastening patch in the first plurality of strap fastening patches mounted to the first detachable movable side flap, the proximal strap end of each strap in the second plurality of straps and each strap fastening patch in the second plurality of strap fastening patches mounted to the second detachable movable side flap, with the straps and loops mounted so that when the first detachable movable side flap is attached to the first side of the base and the second detachable movable side flap is attached to the second side of the base, each strap in said first plurality of straps is opposite a strap fastening patch in said second plurality of strap fastening patches and each strap in said second plurality of straps is opposite a strap fastening patch in said first plurality of strap fastening patches whereby the positioning of the detachable movable side flaps is adjustable by removing the detachable movable side flaps from the base and repositioning them on the base according to the size of the foot and ankle of the intended wearer of the brace, and the interior surface of the distal strap end of each strap may be detachably attached to the exterior surface of a strap fastening patch to apply tension to the strap to secure the base about the foot and ankle.

6. The ankle brace of claim 5 wherein the first, second, third, and fourth detachable attachment means comprise hook and loop type synthetic fastening material.

7. The ankle brace of claim 5 wherein the strap fastening patches and proximal strap ends are attached to the carrier strips along a line which is slightly curved in conformance with the curvature of the front superior surface of the foot and ankle.

8. The ankle brace of claim 5 further comprising enlongate pockets on either side of the base, and a resilient stay member located in each elongate pocket.

9. An adjustable size ankle brace, comprising:
(a) a base of flexible material shaped to wrap around the rear and sides of a foot and ankle, the base having a first side and a second side;
(b) a first reinforcing patch secured to the first side of the base, and a second reinforcing patch secured to the second side of the base;
(c) a first carrier strip secured to the first reinforcing patch and a second carrier strip secured to the second reinforcing patch;
(d) a first plurality of straps and a second plurality of straps, each strap having a proximal strap end with an exterior surface, a first detachable attachment means on the exterior surface of the proximal strap end, a distal strap end with an exterior surface, and second detachable attachment means on the exterior surface of the distal strap end;
(e) a first plurality of loops and a second plurality of loops,
(f) the proximal strap end of each strap in the first plurality of straps and each loop in the first plurality of loops mounted to the first carrier strip, the proximal strap end of each strap in the second plurality of straps and each loop in the second plurality of loops mounted to the second carrier strip, the straps and loops mounted so that each strap in said first plurality of straps is opposite, and trainable through, a loop in said second plurality of loops and each strap in said second plurality of straps is opposite, and trainable through, a loop in said first plurality of loops whereby the distal end of each strap may be trained through a loop and the exterior surface of the distal end of the strap detachably attached to the exterior surface of the proximal end of the strap to adjust the size of the ankle brace and apply tension to the straps to secure the base about the foot and ankle.

10. The ankle brace of claim 9 wherein the first and second detachable attachment means comprise hook and loop type synthetic fastening material.

11. The ankle brace of claim 9 wherein the loops and proximal strap ends are attached to the carrier strips along a line which is slightly curved in conformance with the curvature of the front superior surface of the foot and ankle.

12. The ankle brace of claim 9 further comprising enlongate pockets on either side of the base, and a resilient stay member located in each elongate pocket.

13. An adjustable size ankle brace, comprising:
(a) a base of flexible material shaped to wrap around the rear and sides of a foot and ankle, the base having a first side and a second side;
(b) a first reinforcing patch secured to the first side of the base, and a second reinforcing patch secured to the second side of the base;
(c) a first carrier strip secured to the the first reinforcing patch and a second carrier strip secured to the second reinforcing patch;
(d) a first plurality of straps and a second plurality of straps, each strap having a proximal strap end, a distal strap end with an interior surface, and a first detachable attachment means on the interior surface of the distal strap end;
(e) a first plurality of strap fastening patches and a second plurality of strap fastening patches, each strap fastening patch having an exterior surface and a second detachable attachment means on the exterior surface of the strap fastening patch;
(f) the proximal strap end of each strap in the first plurality of straps and each strap fastening patch in the first plurality of strap fastening patches mounted to the first carrier strip, the proximal strap end of each strap in the second plurality of straps and each strap fastening patch in the second plurality of strap fastening patches mounted to the second carrier strip with the straps and strap fastening patches mounted so that each strap in said first plurality of straps is opposite a strap fastening patch in said second plurality of strap fastening patches and each strap in said second plurality of straps is opposite a strap fastening patch in said first plurality of strap fastening patches whereby the interior surface of the distal end of each strap may be detachably attached to the exterior surface of a strap fastening patch to adjust the size of the ankle brace and apply tension to the straps to secure the base about the foot and ankle.

14. The ankle brace of claim 13 wherein the first and second detachable attachment means comprise hook and loop type synthetic fastening material.

15. The ankle brace of claim 13 wherein the strap fastening patches and proximal strap ends are attached to the carrier strips along a line which is slightly curved in conformance with the curvature of the front superior surface of the foot and ankle.

16. The ankle brace of claim 13 further comprising enlongate pockets on either side of the base, and a resilient stay member located in each elongate pocket.
